(19) United States
(12) Patent Application Publication
(10) Pub. No.: US 2005/0185755 A1
(43) Pub. Date: Aug. 25, 2005

(54) Radiographic apparatus and radiation detection signal processing method

(75) Inventor: Shoichi Okamura, Nara-ken (JP)

Correspondence Address: RADER FISHMAN & GRAUER PLLC, LION BUILDING, 1233 20TH STREET N.W., SUITE 501, WASHINGTON, DC, 20036, US

(73) Assignee: SHIMADZU CORPORATION

(21) Appl. No.: 11/061,783

(22) Filed: Feb. 22, 2005

(30) Foreign Application Priority Data
Feb. 25, 2004 (JP) JP2004-049327

(51) Int. Cl. G21K 1/12; H05G 1/60; A61B 6/00; G01N 23/00

(52) U.S. Cl. 378/22

(57) ABSTRACT

In a radiographic apparatus according to this invention, when an imaging system scan is performed, a imaging system scanner moves an X-ray tube, which emits a cone-shaped X-ray beam, on one linear track, and an FPD, which detects transmission X-ray images of an object under inspection, on the other linear track synchronously with movement of the X-ray tube. Thus, a non-revolving type imaging system scan is carried out. When an X-ray sectional image reconstruction is performed, a sectional image reconstructing unit reconstructs X-ray sectional image from X-ray detection signals of transmission X-ray images of the object detected by the FPD at different radiographic angles. At this time, a time lag remover uses lag-free X-ray detection signals with lag-behind parts removed from the X-ray detection signals. As a result, the lag-behind parts included in the X-ray detection signals, which would cause a lowering of image quality, are removed in advance of a reconstruction of X-ray sectional images.

### BACKGROUND OF THE INVENTION

(1) Field of the Invention

This invention has a radiation emitting device movable on one of two non-circular tracks opposed to each other across an object under inspection for emitting a cone-shaped beam, and a planar radiation image detecting device movable on the other track synchronously with movement of the radiation emitting device for detecting transmission X-ray images of the object. The invention relates to a non-revolving type radiographic apparatus for reconstructing radiation sectional images of the object based on radiation detection signals of transmission radiation images of the object detected from different radiographic angles by the radiation image detecting device in movement. More particularly, the invention relates to a technique for inhibiting a lowering in quality of radiation sectional images caused by lag-behind parts included in the radiation detection signals.

(2) Description of the Related Art

Conventionally, a revolving type X-ray radiographic apparatus or X-ray CT apparatus is installed in medical institutions such as hospitals. The apparatus includes an X-ray tube for emitting a cone-shaped X-ray beam, and an X-ray detector for detecting transmission X-ray images of a patient. The X-ray tube and X-ray detector are arranged to make one complete circle (or at least a semicircle) along a circular track around the patient. Apart from the revolving type apparatus, a non-revolving type X-ray radiographic apparatus also is used.

A non-revolving type X-ray radiographic apparatus, in particular, has an X-ray tube movable on one of two non-circular tracks (e.g. two linear tracks) opposed to each other across a patient for emitting a cone-shaped beam, and a planar X-ray detector movable on the other track synchronously with movement of the X-ray tube for detecting transmission X-ray images of the patient. With the movement of the X-ray tube and X-ray detector, X rays are detected by the X-ray detector at different radiographic angles. The apparatus reconstructs X-ray sectional images of the patient based on radiation detection signals of a plurality of transmission radiation images of the patient.

The non-revolving type X-ray radiographic apparatus, compared with the revolving type apparatus, can perform X-ray radiography without moving the X-ray tube and X-ray detector through more than a semicircle (see Japanese Unexamined Patent Publication No. 2002-263093, page 2 and FIGS. 1 through 3).

As the planar X-ray detector used in the non-revolving type X-ray radiographic apparatus for detecting transmission X-ray images of the patient, a flat panel radiation detector (FPD) has become widely used in recent years, in place of the former type image intensifier.

A mode of reconstructing X-ray sectional images in the conventional non-revolving type X-ray radiographic apparatus will particularly be described with reference to FIG. 1.

A radiographed section Ma of patient M will eventually be displayed in a clear, extracted state as shown in FIG. 1. In X-ray radiography, an X-ray tube 51 is moved horizontally from a right-hand position P1 to a left-hand position P2 in FIG. 1 to change the irradiating angle of X rays emitted from the X-ray tube 51. With changes in the emission angle of X-ray tube 51, an image intensifier tube 52 is moved horizontally from left to right in FIG. 1 to acquire X-ray detection signals of a plurality of transmission X-ray images of the patient having different radiographic angles. An integration process (addition) is carried out to superimpose and compose transmission X-ray images by using the acquired X-ray detection signals.

That is, the image intensifier tube 52 is moved according to the emission angle of X-ray tube 51 so that points A and B located in the radiographed section Ma are constantly projected to corresponding points a and b on the X-ray detecting surface 52a of the image intensifier tube 52. With this construction, a point C outside the radiographed section Ma is projected to varied positions on the X-ray detecting surface 52a as the irradiation angle of X rays changes. At a radiographic angle when the X-ray tube 51 is in the position P1, the point C is projected to a point c1 on the X-ray detecting surface 52a. At a radiographic angle when the X-ray tube 51 has moved to the different position P2, the point C is projected to a point c2 on the X ray detecting surface 52a.

When the acquired X-ray detection signals are integrated, signals from the point C are distributed over entire X-ray sectional images, for example. As a result, the point C in the X-ray sectional images in a fully integrated state becomes a blurred image. The farther away the point C is from the radiographed section Ma, the greater degree of blur occurs. Thus, by integrating the X-ray detection signals of a plurality of transmission X-ray images acquired from different radiographic angles, only the radiographed section Ma appears clearly in the X-ray images composed. That is, the X-ray images obtained present views as if the patient M were incised at the radiographed section Ma.

However, the conventional non-revolving type X-ray radiographic apparatus has a drawback of quality lowering of X-ray sectional images caused by lag-behind parts included in the X-ray detection signals.

That is, a part remaining unread of an X-ray detection signal acquired previously becomes superimposed on a following X-ray detection signal as a lag-behind part or noise (error part). This noise poses a problem of impairing the quality of X-ray sectional images.

### SUMMARY OF THE INVENTION

This invention has been made having regard to the state of the art noted above, and its object is to provide a non-revolving type radiographic apparatus which can inhibit a lowering in quality of radiation sectional images caused by lag-behind parts included in radiation detection signals.

The above object is fulfilled, according to this invention, by a radiographic apparatus for obtaining radiographic images, comprising a radiation emitting device for emitting a cone-shaped radiation beam toward an object under inspection placed on a top board; a planar radiation image detecting device opposed to the radiation emitting device across the object for detecting transmission radiation images of the object; an imaging system scanning device for synchronously moving the radiation emitting device on one of two non-circular tracks opposed to each other across the object, and the radiation image detecting device on the other track; a sectional image reconstructing device for reconstructing radiation sectional images of the object based on radiation detection signals of the transmission radiation images of the object detected from different radiographic angles by the radiation image detecting device while the radiation emitting device and the radiation image detecting device are moved by the imaging system scanning device; and a time lag removing device for obtaining lag-free radiation detection signals by removing lag-behind parts from the radiation detection signals outputted from the radiation image detecting device; wherein the sectional image reconstructing device reconstructs the radiation sectional images by using the lag-free radiation detection signals obtained by the time lag removing device.

According to this invention, when the radiographic apparatus (which may be referred to hereinafter as “tomographic apparatus”) performs a radiographic operation, the imaging system scanning device synchronously moves the radiation emitting device on one of the two non-circular tracks opposed to each other across the object, and moves (scans) the planar radiation image detecting device on the other track. During this scanning movement, the radiation emitting device emits a cone-shaped radiation beam from different emission angles to the object, and the radiation image detecting device detects a plurality of transmission radiographic images of the object. The sectional image reconstructing device reconstructs radiation sectional images based on the radiation detection signals of the transmission radiographic images of the object.

For reconstructing the radiation sectional images, the time lag removing device obtains lag-free radiation detection signals by removing lag-behind parts included in the radiation detection signals outputted from the radiation image detecting device. The sectional image reconstructing device reconstructs the radiation sectional images by using the lag-free radiation detection signals obtained by the time lag removing device.

That is, the radiographic apparatus according to this invention can inhibit a lowering in quality of the radiation sectional images due to the lag-behind parts included in the radiation detection signals.

According to this invention, it is preferable that the sectional image reconstructing device reconstructs the radiation sectional images of the object by performing an integrating process to superimpose and compose transmission radiation images, utilizing the lag-free radiation detection signals obtained by the time lag removing device from the radiation detection signals of the transmission radiation images of the object detected from different radiographic angles.

With this construction, the sectional image reconstructing device can reconstruct the radiation sectional images by a simple data processing, i.e. an integrating process to superimpose and compose transmission radiation images, utilizing the lag-free radiation detection signals obtained from the radiation detection signals of the transmission radiation images of the object detected from different radiographic angles.

The radiographic apparatus according to this invention may further comprise a lag-free radiation signal storage device for successively storing the lag-free radiation detection signals obtained by the time lag removing device from the radiation detection signals of the transmission radiation images of the object detected from different radiographic angles; wherein the sectional image reconstructing device reconstructs the radiation sectional images of the object by performing an integrating process to superimpose and compose transmission radiation images, utilizing the lag-free radiation detection signals successively stored in the lag-free radiation signal storage device.

This construction is effective to inhibit a lowering in quality of the radiation sectional images due to the lag-behind parts included in the radiation detection signals.

The radiographic apparatus may further comprise a signal sampling device for taking the radiation detection signals from the radiation detecting device at predetermined sampling time intervals; wherein the time lag removing device removes the lag-behind parts from the radiation detection signals by a recursive computation, on an assumption that a lag-behind part included in each of the radiation detection signals taken by the signal sampling device at the predetermined sampling time intervals is due to an impulse response formed a plurality of exponential functions with different attenuation time constants.

With this construction, the signal sampling device takes the radiation detection signals from the radiation detecting device at the predetermined sampling time intervals, and the time lag removing device computes the lag-free radiation detection signals by removing the lag-behind parts from the radiation detection signals by a recursive computation. The recursive computation is based on the assumption that a lag-behind part included in each of the radiation detection signals is due to an impulse response formed a plurality of exponential functions with different attenuation time constants. Compared with the case of assuming an impulse response formed of a single exponential function, the lag-behind part is fully removed from each radiation detection signal to produce a lag-free X-ray detection signal.

Specifically, it is preferred that the time lag removing device performs the recursive computation for removing the lag-behind part from each of the radiation detection signals, based on the following equations A-C:
Xₖ=Yₖ−Σₙ₌₁^{N}{αₙ·[1−exp(Tₙ)]·exp(Tₙ)·Sₙₖ} A
Tₙ=−Δt/τₙ B
Sₙₖ=Xₖ₋₁+exp(Tₙ)·Sₙ₍ₖ₋₁₎ C

where

Δt: the sampling time interval;

k: a subscript representing a k-th point of time in a sampling time series;

Yₖ: an X-ray detection signal taken at the k-th sampling time;

Xₖ: a lag-free X-ray detection signal with a lag-behind part removed from the signal Yₖ;

Xₖ₋₁: a signal Xₖtaken at a preceding point of time;

Sₙ₍ₖ₋₁₎: an Sₙat a preceding point of time;

exp: an exponential function;

N: the number of exponential functions with different time constants forming the impulse response;

n: a subscript representing one of the exponential functions forming the impulse response;

αₙ: an intensity of exponential function n; and

τₙ: an attenuation time constant of exponential function n.

The sectional image reconstructing device may reconstruct the radiation sectional images by back projection of projection data resulting from a convolution process, to a set of lattice points virtually set to a section under inspection of the object.

The radiographic apparatus may be a medical apparatus or may be an apparatus for industrial use. In particular, the apparatus for industrial use may be a nondestructive inspecting apparatus.

The planar radiation image detecting device may comprise a flat panel X-ray detector having numerous radiation detecting elements formed of a semiconductor and arranged longitudinally and transversely on a radiation detecting surface.

Where a flat panel X-ray detector is used, the time lag removing device eliminates the time lags in the radiation detection signals provided by the flat panel X-ray detector, and removes complicated detection distortions from output images.

The object noted hereinbefore is fulfilled, according to another aspect of this invention, by a radiation detection signal processing method for taking, at predetermined sampling time intervals, radiation detection signals while synchronously moving a radiation emitting device on one of two non-circular tracks opposed to each other across an object under inspection, and moving a radiation image detecting device on the other track, and performing a signal processing to obtain radiographic images based on the radiation detection signals outputted at the predetermined sampling time intervals, the method comprising the step of removing lag-behind parts from the radiation detection signals by a recursive computation, on an assumption that a lag-behind part included in each of the radiation detection signals taken at the predetermined sampling time intervals is due to an impulse response formed of one exponential function or a plurality of exponential functions with different attenuation time constants.

Specifically, it is preferred that the recursive computation for removing the lag-behind part from each of the radiation detection signals is based on the following equations A-C:
Xₖ=Yₖ−Σₙ₌₁^{N}{αₙ·[1−exp(Tₙ)]·exp(Tₙ)·Sₙₖ} A
Tₙ=−Δt/τₙ B
Sₙₖ=Xₖ₋₁+exp(Tₙ)·Sₙ₍ₖ₋₁₎ C

where

Δt: the sampling time interval;

k: a subscript representing a k-th point of time in a sampling time series;

Yₖ: an X-ray detection signal taken at the k-th sampling time;

Xₖ: a lag-free X-ray detection signal with a lag-behind part removed from the signal Yₖ;

Xₖ₋₁: a signal Xₖtaken at a preceding point of time;

Sₙ₍ₖ₋₁₎: an Sₙat a preceding point of time;

exp: an exponential function;

N: the number of exponential functions with different time constants forming the impulse response;

n: a subscript representing one of the exponential functions forming the impulse response;

αₙ: an intensity of exponential function n; and

τₙ: an attenuation time constant of exponential function n.

### BRIEF DESCRIPTION OF THE DRAWINGS

For the purpose of illustrating the invention, there are shown in the drawings several forms which are presently preferred, it being understood, however, that the invention is not limited to the precise arrangement and instrumentalities shown.

FIG. 1 is a schematic explanatory view showing a mode of reconstructing X-ray sectional images in a conventional apparatus;

FIG. 2 is a block diagram showing an overall construction of an X-ray radiographic apparatus according to the invention;

FIG. 3 is a plan view of an FPD used in the X-ray radiographic apparatus;

FIG. 4 is a schematic view showing a state of sampling X-ray detection signals during X-ray radiography by the apparatus according to the invention;

FIG. 5 is a flow chart showing a recursive computation process for time lag removal in the apparatus according to the invention;

FIG. 6 is a schematic explanatory view showing a mode of reconstructing X-ray sectional images in the apparatus according to the invention;

FIG. 7 is a flow chart showing a radiographic procedure of X-ray radiography in the apparatus according to the invention; and

FIG. 8 is a schematic view showing an outline of a scanning system in a modified X-ray radiographic apparatus.

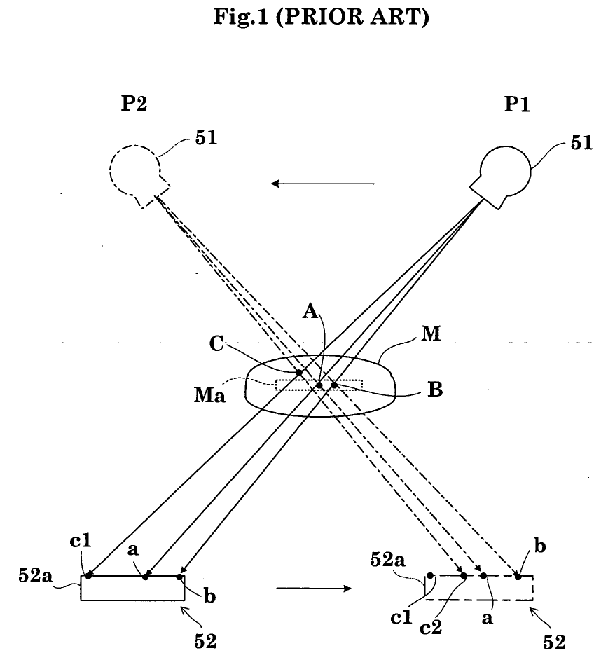

### DESCRIPTION OF THE PREFERRED EMBODIMENTS

A preferred embodiment of this invention will be described in detail hereinafter with reference to the drawings.

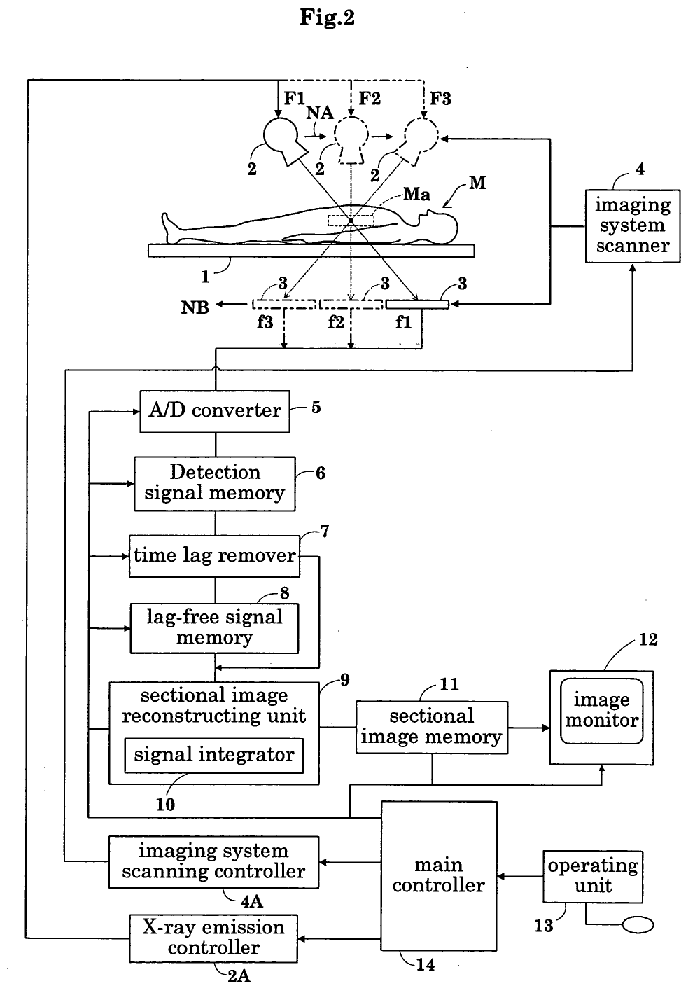

FIG. 2 is a block diagram showing an overall construction of an X-ray radiographic apparatus according to this invention.

As shown in FIG. 2, the X-ray radiographic apparatus includes a top board 1 for supporting a patient M to be radiographed, an X-ray tube 2 acting as a radiation emitting device for emitting a cone-shaped X-ray beam to the patient M on the top board 1, a flat panel X-ray detector 3 (hereinafter referred to as FPD as appropriate) acting as a planar radiation detecting device opposed to the X-ray tube 2 across the patient M for detecting transmission X-ray images of the patient M, and an imaging system scanner 4 acting as an imaging system scanning device for moving the X-ray tube 2 on one linear track NA of two linear tracks NA and NB acting as non-circular tracks opposed to each other across the patient M, and for moving the FPD 3 on the other track NB synchronously with movement of the X-ray tube 2.

When the apparatus in this embodiment performs radiography, the imaging system scanner 4 synchronously moves the X-ray tube 2 on the linear track NA and the FPD 3 on the linear track NB. Thus, while performing a non-revolving type imaging system scan, the X-ray tube 2 is driven to emit a cone-shaped X-ray beam to the patient M from successively varying emission angles. The FPD 3 detects X-ray detection signals of transmission X-ray images of the patient M with different radiographic angles.

Specifically, the imaging system scanner 4 has a function for linearly moving the X-ray tube 2, a function for changing the X-ray emission angle (swing angle) of the X-ray tube 2, and a function for linearly moving the FPD 3. As shown in FIG. 2, the imaging system scanner 4 is operable under control of an imaging system scanning controller 4A for horizontally moving the X-ray tube 2 to a position F1, a position F2 and a position F3 in order, and at the same time adjusting the swing angle of the X-ray tube 2 to change the X-ray emission angle. In accordance with the changes in the X-ray emission angle, the imaging system scanner 4 moves the FPD 3 to a position f1, a position f2 and a position f3 in order, to effect an imaging system scan.

The X-ray tube 2 is operable under control of an X-ray emission controller 2A for emitting a cone-shaped X-ray beam to the patient M at appropriate times.

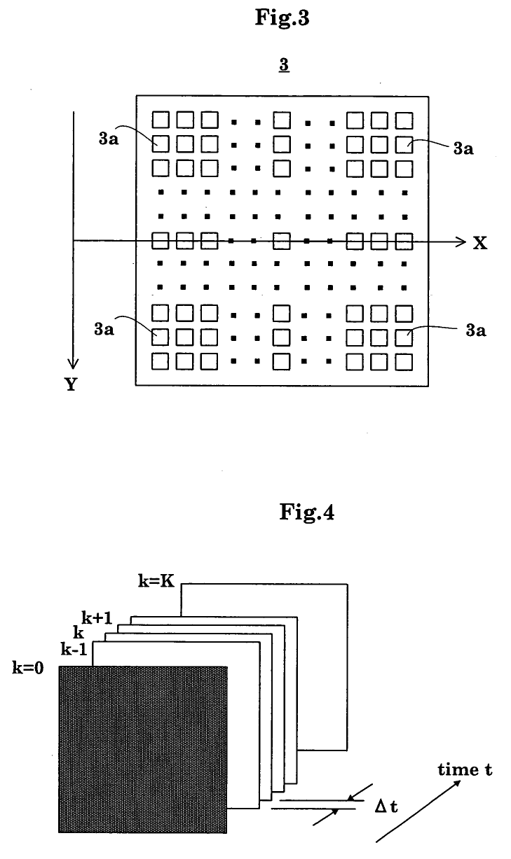

As shown in FIG. 3, the FPD 3 has numerous X-ray detecting elements 3a arranged longitudinally and transversely along the direction X of the body axis of patient M and the direction Y perpendicular to the body axis, on an X-ray detecting surface 3A to which transmission X-ray images from the patient M are projected. In the FPD 3 used in this embodiment, for example, the X-ray detecting elements 3a are arranged to form a matrix of 1,024 by 1,024 on the X-ray detecting surface 3A about 30 cm long and 30 cm wide. Since the FPD 3 is shaped thin and is lightweight, the structure around the FPD 3 is compact. Its flat surface produces little image distortion. As a result, the radiation detection signals accurately correspond to the transmission radiographic images of the patient M.

In the apparatus in this embodiment, the top board 1 is movable by a top board drive mechanism (not shown) vertically as well as longitudinally and transversely. Thus, positions of the X-ray tube 2 and FPD 3 relative to the patient M are variable by movement of the top board 1, thereby making adjustments of a body region under inspection and of radiographic magnification.

As shown in FIG. 2, the X-ray radiographic apparatus in this embodiment further includes, connected to and arranged downstream of the FPD 3, an analog-to-digital converter 5 acting as a signal sampling device for fetching from the FPD 3 and digitizing X-ray detection signals (radiation detection signals) at predetermined sampling time intervals Δt, a detection signal memory 6 for temporarily storing the X-ray detection signals outputted from the analog-to-digital converter 5, a time lag remover 7 for obtaining lag-free X-ray detection signals (lag-free radiation detection signals) by removing lag-behind parts from the X-ray detection signals taken from the FPD 3, and a lag-free signal memory 8 for temporarily storing the lag-free X-ray detection signals having the lag-behind parts removed from the X-ray detection signals. The lag-free signal memory 8 corresponds to the lag-free radiation detection signal storage device of this invention.

The analog-to-digital converter 5 continually fetches the X-ray detection signals of the transmission X-ray images at the sampling time intervals At, and stores the X-ray detection signals in the X-ray detection signal memory 6 disposed downstream of the converter 5. That is, as shown in FIG. 4, all X-ray detection signals for a transmission X-ray image are collected at each period between the sampling intervals Δt, e.g. every 1/30 second, and are successively stored in the X-ray detection signal memory 6.

An operation for sampling (fetching) the X-ray detection signals is started before X-ray irradiation. The sampling of X-ray detection signals by the analog-to-digital converter 5 may be started before an emission of X rays manually by the operator or automatically as interlocked with a command for X-ray emission.

The time lag remover 7 reads the X-ray detection signals from the X-ray detection signal memory 6, and obtains lag-free X-ray detection signals therefrom. A lag-free X-ray detection signal is obtained from each X-ray detection signal by a recursive computation based on an assumption that a lag-behind part included in each X-ray detection signal is due to an impulse response formed of a plurality of exponential functions with different attenuation time constants. The lag-free X-ray detection signals obtained as above are transmitted to the lag-free signal memory 8 and also to a sectional image reconstructing unit 9.

The FPD 3 has part of an X-ray detection signal left unfetched, and this part remains as a lag-behind part in a next X-ray detection signal. The time lag remover 7 removes this lag-behind part to produce a lag-free X-ray detection signal. The time lag remover 7 performs the removing operation based on the assumption that a lag-behind part included in each X-ray detection signal is due to an impulse response formed of a plurality of exponential functions with different attenuation time constants. Compared with the case of assuming an impulse response formed of a single exponential function, the lag-behind part is fully removed from each X-ray detection signal to produce a lag-free X-ray detection signal.

Specifically, the time lag remover 7 performs a recursive computation processing for removing a lag-behind part from each X-ray detection signal by using equations A-C set out hereunder.

As shown in FIG. 2 and in equations A-C, in obtaining a current lag-free X-ray detection signal, the time lag remover 7 performs the recursive computation processing by using a lag-free X-ray detection signal obtained at a preceding point of time and temporarily stored in the lag-free signal memory 8.
Xₖ=Yₖ−Σₙ₌₁^{N}{αₙ·[1−exp(Tₙ)]·exp(Tₙ)·Sₙₖ} A
Tₙ=−Δt/τₙ B
Sₙₖ=Xₖ₋₁+exp(Tₙ)·Sₙ₍ₖ₋₁₎ C

where

Δt: the sampling time interval;

k: a subscript representing a k-th point of time in a sampling time series;

Yₖ: an X-ray detection signal taken at the k-th sampling time;

Xₖ: a lag-free X-ray detection signal with a lag-behind part removed from the signal Yₖ;

Xₖ₋₁: a signal Xₖ taken at a preceding point of time;

Sₙ₍ₖ₋₁₎: an Sₙ at a preceding point of time;

exp: an exponential function;

N: the number of exponential functions with different time constants forming the impulse response;

n: a subscript representing one of the exponential functions forming the impulse response;

αₙ: an intensity of exponential function n; and

τₙ: an attenuation time constant of exponential function n.

That is, the second term in equation A “Σₙ₌₁^{N}{αₙ·[1−exp(Tₙ)]·exp(Tₙ)·Sₙₖ}” corresponds to the lag-behind part. Thus, the apparatus in this embodiment derives the lag-free X-ray detection signal Xₖpromptly from equations A-C constituting a compact recurrence formula.

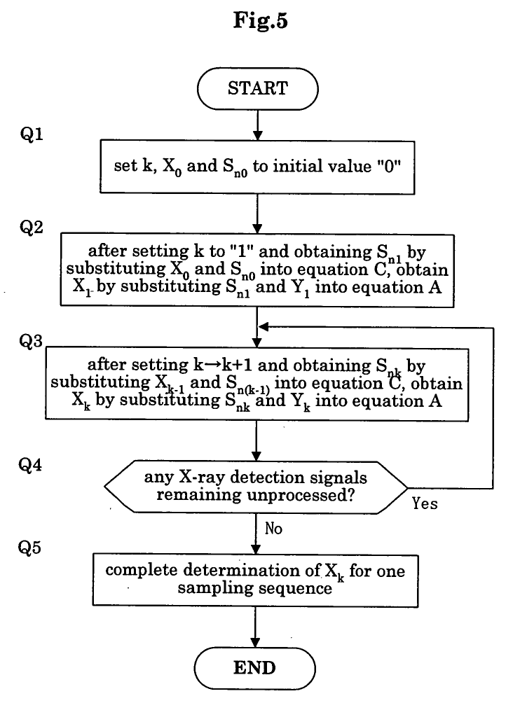

Next, the process of recursive computation carried out by the time lag remover 7 will particularly be described with reference to FIG. 5.

FIG. 5 is a flow chart showing a recursive computation process for time lag removal in this embodiment.

[Step Q1] A setting k=0 is made, and X₀=0 in equation A and Sₙ₀=0 in equation C are set as initial values before X-ray emission. Where the number of exponential functions is three (N=3), S₁₀, S₂₀ and S₃₀ are all set to 0.

[Step Q2] In equations A and C, k=1 is set. S₁₁, S₂₁ and S₃₁ are derived from equation C, i.e. Sₙ₁=X₀+exp(Tₙ) Sₙ₀. Further, lag-free X-ray detection signal X₁ is obtained by substituting S₁₁, S₂₁ and S₃₁ derived and X-ray detection signal Y₁ into equation A.

[Step Q3] After incrementing k by 1 (k=k+1) in equations A and C, S₁ₖ, S₂ₖ and S₃ₖ are obtained by substituting Xₖ₋₁of a preceding time into equation C. Further, lag-free X-ray detection signal Xₖ is obtained by substituting S₁ₖ, S₂ₖ and S₃ₖ derived and X-ray detection signal Yₖ into equation A.

[Step Q4] When there remain unprocessed X-ray detection signals Yₖ, the operation returns to step Q3. When no unprocessed X-ray detection signals Yₖ remain, the operation proceeds to step Q5.

[Step Q5] Lag-free X-ray detection signals Xₖ for one sampling sequence (for one X-ray image) are obtained to complete the recursive computation for the one sampling sequence.

In this embodiment, the time lag remover 7 obtains lag-free X-ray detection signals by using X-ray detection signals taken by the analog-to-digital converter 5 before X-ray emission. Consequently, in time of the X-ray emission, lag-free X-ray detection signals may properly be obtained immediately upon X-ray emission by removing lag-behind parts included in the X-ray detection signals.

As shown in FIG. 2, the X-ray radiographic apparatus in this embodiment includes the sectional image reconstructing unit 9 downstream of the time lag remover 7. The sectional image reconstructing unit 9 reconstructs X-ray sectional images of the patient M based on the X-ray detection signals of a plurality of transmission X-ray images of the patient M detected by the FPD 3 continuously or intermittently at different radiographic angles as the X-ray tube 2 and FPD 3 are moved by the imaging system scanner 4.

Specifically, the sectional image reconstructing unit 9 reconstructs X-ray sectional images, with a signal integrator 10 performing an integrating process to superimpose and compose the lag-free X-ray detection signals obtained by the time lag remover 7 from the X-ray detection signals of transmission X-ray images of the patient M detected at different radiographic angles.

The X-ray sectional images reconstructed by the sectional image reconstructing unit 9 are transmitted to and stored in a sectional image memory 11. The X-ray sectional images are displayed on an image monitor 12, or printed on sheets by a printer (not shown), as necessary.

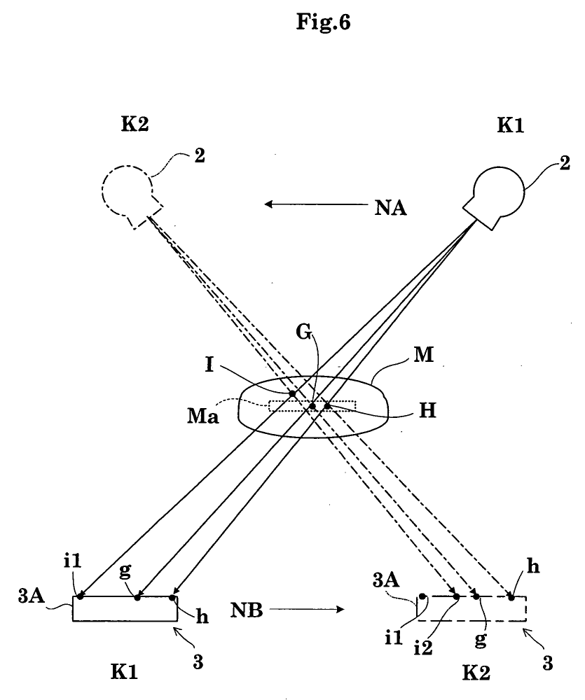

A mode of reconstructing X-ray sectional images in the non-revolving type X-ray radiographic apparatus in this embodiment will particularly be described with reference to FIG. 6.

A radiographed section Ma of the patient M will eventually be displayed in a clear, extracted state. In X-ray radiography, X-ray detection signals of a plurality of transmission X-ray images of the patient M are acquired at different radiographic angles while varying the X-ray emission angle of the X-ray tube 2 and varying the position of the FPD 3 as interlocked with the variations in the X-ray emission angle of the X-ray tube 2. The X-ray detection signals are integrated (added) to superimpose and compose the transmission X-ray images.

Specifically, the FPD 3 is moved according to the emission angle of X-ray tube 2 so that points G and H located in the radiographed section Ma are constantly projected to corresponding points g and h on the X-ray detecting surface 3A of the FPD 3. Then, a point I outside the radiographed section Ma is projected to varied positions on the X-ray detecting surface 3A as the irradiation angle of X rays changes. Δt a radiographic angle when the X-ray tube 2 is in a position K1, the point I is projected to a point il on the X-ray detecting surface 3A in a position k1. At a radiographic angle when the X-ray tube 2 has moved to a different position K2, the point I is projected to a point i2 on the X ray detecting surface 3A in a position k2.

When the X-ray detection signals are integrated, signals from the point I are distributed over entire X-ray sectional images. As a result, the point I in the X-ray sectional images in a fully integrated state becomes a blurred image. The farther away the point I is from the radiographed section Ma, the greater degree of blur occurs. Thus, by integrating the X-ray detection signals of a plurality of transmission X-ray images acquired from different radiographic angles, only the radiographed section Ma appears clearly in the X-ray images composed. That is, the X-ray images obtained present views as if the patient M were incised at the radiographed section Ma.

Thus, according to the apparatus in this embodiment, X-ray sectional images can be reconstructed through a simple data processing carried out by the signal integrator 10 of the sectional image reconstructing unit 9 to integrate the lag-free X-ray detection signals.

The apparatus in this embodiment includes also an operating unit 13 for inputting instructions, data and the like required for executing radiography. This operating unit 13 is in the form of input devices such as a keyboard and a mouse.

In the apparatus in this embodiment, the X-ray emission controller 2A, imaging system scanning controller 4A, analog-to-digital converter 5, time lag remover 7 and sectional image reconstructing unit 9 perform controls and processes according to various commands transmitted from a main controller 14 in response to instructions and data inputted from the operating unit 13 or with progress of a radiographic operation.

Next, an operation for performing X-ray radiography with the apparatus in the embodiment will particularly be described with reference to the drawings.

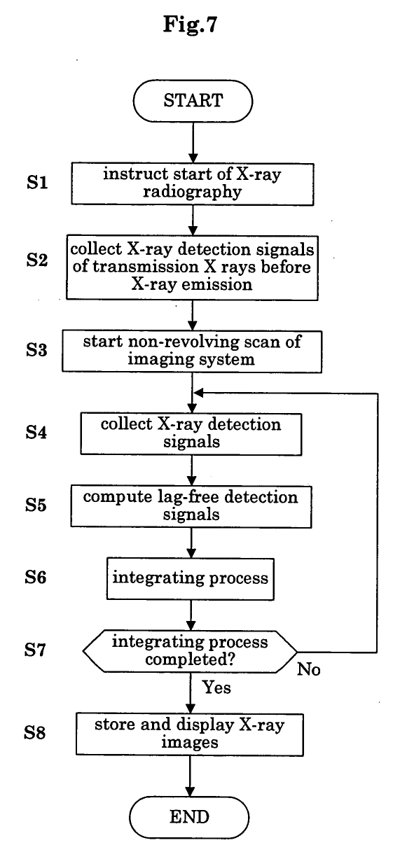

FIG. 7 is a flow chart showing a procedure of X-ray radiography in the embodiment.

[Step S1] The operator, by using the operating unit 13, inputs instructions to start a radiographic operation.

[Step S2] The analog-to-digital converter 3 starts taking X-ray detection signals Yₖfor one X-ray image from the FPD 3 at each period between the sampling time intervals Δt (= 1/30 second) before X-ray emission. The X-ray detection signals taken are stored in the X-ray detection signal memory 6.

[Step S3] In response to settings made by the operator, the imaging system scanner 4 starts a non-revolving imaging system scan to move synchronously the X-ray tube 2 on the linear track NA and the FPD 3 on the linear track NB.

[Step S4] In parallel with an intermittent or continuous X-ray emission to the patient M initiated by the operator, the analog-to-digital converter 5 repeats taking X-ray detection signals Yₖ for one X-ray image at each period between the sampling time intervals At and storing the signals in the X-ray detection signal memory 6.

[Step S5] X-ray detection signals Yₖfor one transmission X-ray image after another are read from the X-ray detection signal memory 6. The time lag remover 7 obtains lag-free X-ray detection signals Xₖ with lag-behind parts removed from the X-ray detection signals Yₖ through recursive computations utilizing the equations A-C. A process is repeated to store the lag-free X-ray detection signals Xₖ in the lag-free signal memory 8.

[Step S6] The signal integrator 10 of the sectional image reconstructing unit 9 performs every moment an integrating process of (i.e. adds) the lag-free X-ray detection signals Xₖ stored in the lag-free signal memory 8, to compose transmission X-ray images.

[Step S7] Until completion of the imaging system scan by the imaging system scanner 4 and the integrating process by the signal integrator 10, the processes in steps S4 to S6 are continued. When the imaging system scan by the imaging system scanner 4 and the integrating process by the signal integrator 10 are completed, it means that X-ray sectional images have been made for the radiographed section Ma. The operation moves to step S8.

[Step S8] The X-ray images of the radiographed section Ma are stored in the sectional image memory 11, and are displayed on the image monitor 12, or printed on sheets by the printer (not shown), as necessary. Then, the radiographic operation is ended.

According to the X-ray radiographic apparatus in this embodiment, as described above, when an imaging system scan is performed, the imaging system scanner 4 moves the X-ray tube 2, which emits a cone-shaped X-ray beam, on one linear track NA of the two linear tracks NA and NB opposed to each other across the patient M, and moves the FPD 3, which detects transmission X-ray images of the patient M, on the other track NB synchronously with movement of the X-ray tube 2. Thus, a non-revolving type imaging system scan is carried out. When an X-ray sectional image reconstruction is performed, and the sectional image reconstructing unit 9 reconstructs X-ray sectional image from the X-ray detection signals of transmission X-ray images of the patient M detected by the FPD 3 continuously or intermittently from different radiographic angles, the time lag remover 7 uses lag-free X-ray detection signals with the lag-behind parts removed from the X-ray detection signals. As a result, the lag-behind parts included in the X-ray detection signals, which would cause a lowering of image quality, are removed in advance of a reconstruction of X-ray sectional images.

Thus, the non-revolving type X-ray radiographic apparatus according to this invention can inhibit a lowering in quality of X-ray sectional images due to the lag-behind parts included in the X-ray detection signals.

This invention is not limited to the foregoing embodiment, but may be modified as follows:

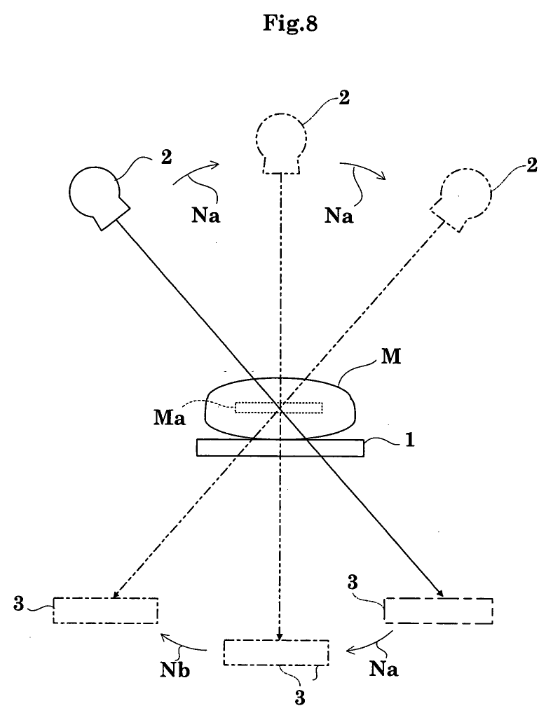

(1) In the foregoing embodiment, the two non-circular tracks opposed to each other across the patient M are the linear tracks NA and NB. Instead, as shown in FIG. 8, the non-circular tracks may be in the form of arcuate tracks Na and Nb.

(2) The foregoing embodiment uses the FPD 3 as the planar radiation detecting device. Instead of the FPD, an image intensifier may be used.

(3) In the foregoing embodiment, the sectional image reconstruction carried out by the sectional image reconstructing unit 9 is in the form of the integrating process by the signal integrator 10. The sectional image reconstructing unit 9 may carry out a sectional image reconstruction, for example, by back projection of projection data produced from lag-free X-ray detection signals Xₖ put to a convolution process, to a set of lattice points virtually set to the section under inspection of the patient M.

(4) In the foregoing embodiment, a non-revolving type imaging system scan is carried out by moving the X-ray tube 2 and FPD 3 linearly. This feature may be modified to adopt other moving modes of the X-ray tube 2 and FPD 3 such as swirling movement, elliptical movement and so on.

(5) The apparatus in the described embodiment is designed for medical use. This invention is applicable not only to such medical apparatus but also to an apparatus for industrial use such as a nondestructive inspecting apparatus.

(6) The apparatus in the described embodiment uses X rays as radiation. This invention is applicable also to an apparatus using radiation other than X rays.

This invention may be embodied in other specific forms without departing from the spirit or essential attributes thereof and, accordingly, reference should be made to the appended claims, rather than to the foregoing specification, as indicating the scope of the invention.

1. A radiographic apparatus for obtaining radiographic images, comprising:
radiation emitting means for emitting a cone-shaped radiation beam toward an object under inspection placed on a top board;
planar radiation image detecting means opposed to said radiation emitting means across said object for detecting transmission radiation images of said object;
imaging system scanning means for synchronously moving said radiation emitting means on one of two non-circular tracks opposed to each other across said object, and said radiation image detecting means on the other track;
sectional image reconstructing means for reconstructing radiation sectional images of said object based on radiation detection signals of the transmission radiation images of the object detected from different radiographic angles by said radiation image detecting means while said radiation emitting means and said radiation image detecting means are moved by said imaging system scanning means; and
time lag removing means for obtaining lag-free radiation detection signals by removing lag-behind parts from the radiation detection signals outputted from said radiation image detecting means;
wherein said sectional image reconstructing means reconstructs the radiation sectional images by using the lag-free radiation detection signals obtained by said time lag removing means.

2. A radiographic apparatus as defined in claim 1, wherein said sectional image reconstructing means reconstructs the radiation sectional images of said object by performing an integrating process to superimpose and compose transmission radiation images, utilizing the lag-free radiation detection signals obtained by the time lag removing means from the radiation detection signals of the transmission radiation images of said object detected from different radiographic angles.

3. A radiographic apparatus as defined in claim 1, further comprising:
lag-free radiation signal storage means for successively storing the lag-free radiation detection signals obtained by said time lag removing means from the radiation detection signals of the transmission radiation images of said object detected from different radiographic angles;
wherein said sectional image reconstructing means reconstructs the radiation sectional images of said object by performing an integrating process to superimpose and compose transmission radiation images, utilizing the lag-free radiation detection signals successively stored in said lag-free radiation signal storage means.

4. A radiographic apparatus as defined in claim 1, further comprising:
signal sampling means for taking the radiation detection signals from said radiation detecting means at predetermined sampling time intervals;
wherein said time lag removing means removes the lag-behind parts from the radiation detection signals by a recursive computation, on an assumption that a lag-behind part included in each of said radiation detection signals taken by said signal sampling means at the predetermined sampling time intervals is due to an impulse response formed of one exponential function or a plurality of exponential functions with different attenuation time constants.

5. A radiographic apparatus as defined in claim 1, wherein said time lag removing means performs a recursive computation for removing the lag-behind part from each of the radiation detection signals, based on the following equations A-C:
Xₖ=Yₖ−Σₙ₌₁^{N}{αₙ·[1−exp(Tₙ)]·exp(Tₙ)·Sₙₖ} A
Tₙ=−Δt/τₙ B
Sₙₖ=Xₖ₋₁+exp(Tₙ)·Sₙ₍ₖ₋₁₎ C
where
Δt: the sampling time interval;
k: a subscript representing a k-th point of time in a sampling time series;
Yₖ: an X-ray detection signal taken at the k-th sampling time;
Xₖ: a lag-free X-ray detection signal with a lag-behind part removed from the signal Yₖ;
Xₖ₋₁: a signal Xₖ taken at a preceding point of time;
Sₙ₍ₖ₋₁₎: an Sₙ at a preceding point of time;
exp: an exponential function;
N: the number of exponential functions with different time constants forming the impulse response;
n: a subscript representing one of the exponential functions forming the impulse response;
αₙ: an intensity of exponential function n; and
τₙ: an attenuation time constant of exponential function n.

6. A radiographic apparatus as defined in claim 1, wherein said sectional image reconstructing means reconstructs the radiation sectional images by back projection of projection data resulting from a convolution process, to a set of lattice points virtually set to a section under inspection of said object.

7. A radiographic apparatus as defined in claim 1, wherein said planar radiation image detecting means comprises a flat panel X-ray detector having numerous radiation detecting elements formed of a semiconductor and arranged longitudinally and transversely on a radiation detecting surface.

8. A radiographic apparatus as defined in claim 1, wherein said apparatus is a medical apparatus.

9. A radiographic apparatus as defined in claim 1, wherein said apparatus is for industrial use.

10. A radiographic apparatus as defined in claim 9, wherein said apparatus for industrial use comprises a nondestructive inspecting apparatus.

11. A radiation detection signal processing method for taking, at predetermined sampling time intervals, radiation detection signals while synchronously moving radiation emitting means on one of two non-circular tracks opposed to each other across an object under inspection, and moving radiation image detecting means on the other track, and performing a signal processing to obtain radiographic images based on the radiation detection signals outputted at the predetermined sampling time intervals, said method comprising the step of:
removing lag-behind parts from the radiation detection signals by a recursive computation, on an assumption that a lag-behind part included in each of said radiation detection signals taken at the predetermined sampling time intervals is due to an impulse response formed of one exponential function or a plurality of exponential functions with different attenuation time constants.

12. A radiation detection signal processing method as defined in claim 11, wherein said recursive computation for removing the lag-behind part from each of the radiation detection signals is based on the following equations A-C:
Xₖ=Yₖ−Σₙ₌₁^{N}{αₙ·[1−exp(Tₙ)]·exp(Tₙ)·Sₙₖ} A
Tₙ=−Δt/τₙ B
Sₙₖ=Xₖ₋₁+exp(Tₙ)·Sₙ₍ₖ₋₁₎ C
where Δt: the sampling time interval;
k: a subscript representing a k-th point of time in a sampling time series;
Yₖ: an X-ray detection signal taken at the k-th sampling time;
Xₖ: a lag-free X-ray detection signal with a lag-behind part removed from the signal Yₖ;
Xₖ₋₁: a signal Xₖ taken at a preceding point of time;
Sₙ₍ₖ₋₁₎: an Sₙ at a preceding point of time;
exp: an exponential function;
N: the number of exponential functions with different time constants forming the impulse response;
n: a subscript representing one of the exponential functions forming the impulse response;
αₙ: an intensity of exponential function n; and
τₙ: an attenuation time constant of exponential function n.
